(19) United States
(12) Patent Application Publication
(10) Pub. No.: US 2022/0288060 A1
(43) Pub. Date: Sep. 15, 2022

(54) Treatment of Opioid Withdrawal

(71) Applicant: Kinoxis Therapeutics Pty Ltd, Camberwell (AU)

(72) Inventors: Michael Thomas BOWEN, Camberwell (AU); Iain Stewart MCGREGOR, Camberwell (AU)

(21) Appl. No.: 17/637,512

(22) PCT Filed: Sep. 7, 2020
(86) PCT No.: PCT/AU2020/050941

§ 371 Date: Feb. 23, 2022

(30) Foreign Application Priority Data
Sep. 6, 2019 (AU) 2019903299

(51) Int. Cl. A61K 31/485; A61P 25/36; A61K 31/5517; A61K 45/06

(52) U.S. Cl. CPC A61K 31/485; A61P 25/36; A61K 31/5517; A61K 45/06

(57) ABSTRACT

This invention relates to methods of treating, preventing, managing and/or controlling opioid withdrawal and/or a symptom associated with the opioid withdrawal. Compounds, compositions, medicaments and kits are provided that may be used in these methods.

### CROSS-REFERENCE TO RELATED APPLICATION(S)

The present application claims priority from Australian Provisional Patent Application 2019903299 filed on 6 Sep. 2019, the entire contents of which is incorporated herein by reference.

### FIELD OF THE INVENTION

This invention relates to methods for treating and/or preventing opioid withdrawal.

### BACKGROUND OF THE INVENTION

Opioids and opiates are a useful class of analgesics that find widespread use in pain management. Opioids and opiates have also become substances of abuse—both by recreational users and by patients who develop opioid use disorder following opioid therapy. Cessation of opioid and/or opiate use may also lead to opioid withdrawal. Opioid withdrawal is a physiological condition resulting from a subject's physical dependence on the opioid and/or opiate and in some instances can develop after exposure to opioids and/or opiates for short periods of time.

For example, iatrogenic opioid withdrawal syndrome (IOWS) frequently emerges in patients who have undergone or are undergoing treatment with opioid analgesics for acute or chronic pain management. For instance, it was reported in 2017 that 16.7% of adults in an intensive care unit (ICU) receiving on average ˜6 days of opioid treatment suffered IOWS. It has also been reported that in pediatric populations, 10-57% of patients receiving opioids in the ICU for more than 24 h suffer IOWS. The proportion of patients suffering IOWS increases drastically when opioid treatment is longer-term. For instance, some randomized controlled trials (RCTs) reported over 80% of patients suffering IOWS upon opioid dose tapering or discontinuation of an opioid. IOWS presents a major challenge for treating physicians as it can prevent successful discontinuation of opioid use, drastically increasing the risk of treatment-related adverse events and opioid misuse. In some populations as many as ⅔ patients fail to successfully discontinue opioid treatment, with withdrawal symptoms a major factor contributing to this failure.

Opioid withdrawal causes significant pain, physical and psychological distress to sufferers. Therefore, providing effective treatments for opioid withdrawal is desirable to provide acute relief for subjects in need thereof. Further, desire to escape opioid withdrawal symptoms plays a role in patients transitioning from as prescribed use to abuse, thus effectively preventing or treating opioid withdrawal may help prevent the development of an opioid use disorder in an individual who may have initially used opioid and/or opiates in a prescribed manner. Also, overcoming opioid withdrawal symptoms is typically the first major hurdle to recovery in individuals suffering from an opioid use disorder. Therefore, effectively treating opioid withdrawal may assist a subject achieve sobriety.

In the context of opioid use disorder, existing management strategies for opioid withdrawal include replacement therapy, where an opioid (typically buprenorphine or methadone) is administered as a replacement opioid. Replacement opioid therapy seeks to prevent or delay the emergence of opioid withdrawal and/or minimise the severity of the withdrawal syndrome that emerges. However, the opioids used for replacement therapy often themselves subsequently result in opioid withdrawal, along with other side-effects, when they are discontinued or their dose is tapered. Further, replacement therapy may be required for long periods of time, especially if the subject has developed opioid use disorder.

Lofexidine was the first, and at the time of filing remains the only, non-opioid drug approved by the US Food and Drug Administration (FDA) for the management of opioid withdrawal symptoms. However, in clinical trials lofexidine therapy resulted in only modest improvements in acute opioid withdrawal symptoms and treatment retention, while causing concerning side effects, including hypotension, bradycardia and insomnia. Moreover, lofexidine is only approved for use for a maximum of 14 days.

There is therefore a continuing need to provide alternative options for managing opioid withdrawal.

All publications, patents and patent applications that may be cited herein are hereby incorporated by reference in their entirety.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The invention provides a method of treating opioid withdrawal and/or a symptom associated with the opioid withdrawal, comprising administering to a subject in need thereof an effective amount of a compound according to Formula (I)

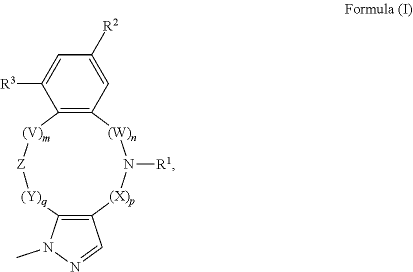

wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.
or a pharmaceutically acceptable salt or prodrug thereof, to thereby treat the opioid withdrawal and/or the symptom associated with the opioid withdrawal in the subject.

The compound may be:

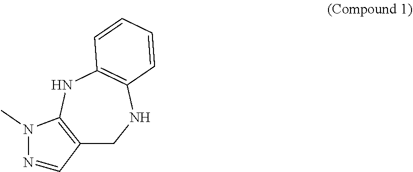

or a pharmaceutically acceptable salt or prodrug thereof.

In some embodiments, the compound may be a hydrochloride salt of Compound 1, such as the di-hydrochloride salt (CMPD1-2HCL).

In some embodiments, the compound may be a phosphoric acid addition salt of Compound 1 (CMPD1-PO4). The phosphoric acid addition salt may be referred to as a phosphate salt of Compound 1.

In another aspect, there is provided a method of treating a subject that has been exposed to an opioid and/or opiate or is at risk of being exposed to an opioid and/or opiate, the method comprising administering to the subject an effective amount of a compound of formula (I) or a pharmaceutically acceptable salt or prodrug thereof to thereby treat and/or prevent opioid withdrawal and/or a symptom associated with the opioid withdrawal.

In another aspect, there is provided use of a compound of formula (I) or a pharmaceutically acceptable salt or prodrug thereof in the manufacture of a medicament for treating opioid withdrawal and/or a symptom associated with the opioid withdrawal.

In another aspect, there is provided use of a compound of formula (I) or a pharmaceutically acceptable salt or prodrug thereof for treating opioid withdrawal and/or a symptom associated with the opioid withdrawal.

In another aspect, there is provided a compound of formula (I) or a pharmaceutically acceptable salt or prodrug thereof for use in treating opioid withdrawal and/or a symptom associated with the opioid withdrawal.

In another aspect, there is provided a pharmaceutical composition comprising an effective amount of a compound of Formula (I) or a pharmaceutically acceptable salt and/or prodrug thereof, and an opioid and/or opiate.

In another aspect, there is provided a pharmaceutical composition comprising an effective amount of a compound of Formula (I) or a pharmaceutically acceptable salt and/or prodrug thereof, and an effective amount of an opioid antagonist and/or partial agonist.

In another aspect, there is provided a kit comprising in separate parts:
an effective amount of a compound of Formula (I) or a pharmaceutically acceptable salt and/or prodrug thereof, and
an opioid and/or opiate.

In another aspect, there is provided a kit comprising in separate parts:
an effective amount of a compound of Formula (I) or a pharmaceutically acceptable salt and/or prodrug thereof, and
an effective amount of an opioid antagonist and/or partial agonist.

In some embodiments, these pharmaceutical compositions and kits may be used in any of the methods described herein.

In another aspect, there is provided a method of preventing opioid withdrawal and/or a symptom associated with the opioid withdrawal, comprising administering to a subject in need thereof an effective amount of a compound of Formula (I) or a pharmaceutically acceptable salt and/or prodrug thereof.

In another aspect, there is provided a method of treating pain, comprising administering to a subject in need thereof an effective amount of an opioid and/or opiate, and an effective amount of a compound of Formula (I) or a pharmaceutically acceptable salt or prodrug thereof. In some embodiments, the dose of the opioid and/or opiate may be reduced according to a tapering regimen. Administration of the compound of Formula (I) may be maintained after cessation of administration of the opioid and/or opiate according to any of the methods of treating, preventing, managing and/or controlling opioid withdrawal and/or a symptom associated with the opioid withdrawal described herein.

In another aspect, there is provided a method of treating opioid overdose, comprising administering to a subject in need thereof an effective amount of an opioid antagonist and an effective amount of a compound of Formula (I) of a pharmaceutically acceptable salt and/or prodrug thereof.

As used herein, except where the context requires otherwise, the term “comprise” and variations of the term, such as “comprising”, “comprises” and “comprised”, are not intended to exclude further additives, components, integers or steps.

It must be noted that as used herein and in the appended claims, the singular forms “a”, “an” and “the” include plural reference unless the context clearly dictates otherwise. Thus, for example, a reference to “a symptom” and/or “at least one symptom” may include one or more symptoms, and so forth.

The term “and/or” can mean “and” or “or”.

The term “(s)” following a noun contemplates the singular or plural form, or both.

Various features of the invention are described with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within ±25%, ±10%, ±5%, ±1% or 20±0.1% of that value.

Unless otherwise herein defined, chemical terms will have their general meanings known in the art.

As used herein, the term “C₁₋₅ alkyl” either used alone or in compound terms, refers to monovalent straight chain or branched hydrocarbon groups, having 1 to 5 carbon atoms. As understood by a person skilled in the art, the term “C₁₋₅ alkyl” means an alkyl chain with 1, 2, 3, 4 or 5 carbon atoms or a range comprising any of two of those integers including 1-2, 1-3, 1-4, 1-5, 2-3, 2-4, 2-5, 3-4, 3-5 and 4-5. Suitable alkyl groups include, but are not limited to: methyl, ethyl, propyl, isopropyl, n-butyl, sec-butyl, tert-butyl, n-pentyl, neopentyl, iso-pentyl and tert-pentyl. The C₁₋₄ alkyl may be optionally substituted with one or more substituents. The substituents may be in any position of the carbon chain. Suitable substituents include, but are not limited to: OH, NH2, halogen, NH(C₁₋₅alkyl), N(C₁₋₅ alkyl)₂, CN, NO₂, CO₂H, or OC₁₋₅alkyl.

The terms “hydroxy” and “hydroxyl” refer to the group —OH.

As used herein, the term “OC₁₋₅ alkyl” either used alone or in compound terms, refers to alkoxy groups having 1 to 5 carbon atoms. As understood by a person skilled in the art, the term “OC₁₋₅ alkyl” means an alkoxy group with 1, 2, 3, 4 or 5 carbon atoms or a range comprising any of two of those integers and including 1-2, 1-3, 1-4, 1-5, 2-3, 2-4, 2-5, 3-4, 3-5 and 4-5. Suitable OC₁₋₅ alkyl groups include, but are not limited to, methoxy, ethoxy, propyloxy, isopropyloxy, n-butyloxy, sec-butyloxy, tert-butyloxy, n-pentyloxy, neopentyloxy, iso-pentyloxy and tert-pentyloxy. The C₁₋₅ alkyl may be optionally substituted with one or more substituents. The substituents may be in any position of the carbon chain. Suitable substituents include, but are not limited to: OH, NH2, halogen, NH(C₁₋₅ alkyl), N(C₁₋₅ alkyl)₂, CN, NO₂, CO₂H, or OC₁₋₅alkyl.

As used herein, the term “halo” or “halogen” refers to fluorine (fluoro), chlorine (chloro), bromine (bromo) or iodine (iodo).

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 is a chart of distance travelled by C57BL/6 mice over time following treatment with CMPD1-2HCL (results of experiment 1 of Example 1).

FIG. 2A is a chart of frequency of jumping by treatment group (0, 2.5, 5 or 10 mg/kg CMPD1-2HCL) after oxycodone withdrawal was precipitated in C57BL/6 mice by naloxone administration (rightmost four groups) and in C57BL/6 mice not undergoing oxycodone withdrawal (leftmost group) (jumping results of experiment 1.2 of Example 1).

FIG. 2B is a chart of duration of paw tremors (duration in seconds) by treatment group (0, 2.5, 5 or 10 mg/kg CMPD1-2HCL) after oxycodone withdrawal was precipitated in C57BL/6 mice by naloxone administration (rightmost four groups) and in C57BL/6 mice not undergoing oxycodone withdrawal (leftmost group) (paw tremor results of experiment 1.2 of Example 1).

FIGS. 3a-h show a series of charts of the number of c-fos positive cells across 8 brain regions (a. the medial division of the central amygdala; b. lateral parabrachial nucleus, c. periaqueductal grey; d. lateral habenula; e. nucleus accumbens shell; f. ventral division of the lateral septum; g. ventral tegmental area; and h. dorsal raphe) by treatment group after oxycodone withdrawal was precipitated in C57BL/6 mice by naloxone administration (rightmost two groups) and in C57BL/6 mice not undergoing oxycodone withdrawal (leftmost two groups) (c-fos results of Example 2).

FIG. 4 is a chart of number of fecal boli by treatment group (vehicle only, oxycodone and oxycodone followed by CMPD1-2HCL) after oxycodone withdrawal was precipitated in C57BL/6 mice by naloxone administration (fecal boli results of Example 3).

FIG. 5 is a chart of frequency of jumping by treatment group (vehicle only, oxycodone, oxycodone followed by CMPD1-PO4 and oxycodone followed by CMPD1-2HCL) after oxycodone withdrawal was precipitated in C57BL/6 mice by naloxone administration in the rightmost three groups (jumping results of Example 4).

### DETAILED DESCRIPTION OF THE EMBODIMENTS

The invention provides a method of treating opioid withdrawal and/or a symptom associated with the opioid withdrawal. The method comprises administering an effective amount of a compound of Formula (I) or a pharmaceutically acceptable salt and/or prodrug thereof.

Surprisingly, the inventors found that a compound of the invention was able to treat symptoms of opioid withdrawal. Therefore, therapy involving administration of a compound of Formula (I) may be useful to treat, prevent, manage and/or control opioid withdrawal and/or a symptom associated with the opioid withdrawal.

Opioid withdrawal is a physiological phenomenon that may emerge after a subject has been exposed to an opioid and/or opiate and has become physically dependent on the opioid and/or opiate. Opioid withdrawal may therefore manifest in subjects when exposure to the opioid and/or opiate is removed, and/or upon exposure to an opioid antagonist and/or opioid partial agonist. There are numerous diagnostic tools that have been developed relating to opioid withdrawal. One such diagnostic tool is defined in the Diagnostic and Statistical Manual of Mental Disorders 5 (DSM-5 or DSM-V). According to DSM-5, opioid withdrawal may be diagnosed by either (1) cessation of (or reduction in) opioid or opiate use that has been heavy and prolonged (ie several weeks or longer); and/or (2) administration of an opioid antagonist after a period of opioid and/or opiate use. However, there are some notable instances where opioid withdrawal has been documented that are not covered by the DSM-5 diagnostic criteria, including neonatal opioid withdrawal and some cases of IOWS.

In the methods of the invention, the subject may have been exposed to an opioid and/or opiate, or is at risk of being exposed to an opioid and/or opiate. In some embodiments, the subject has been administered an opioid and/or opiate. In some embodiments, the subject has been exposed to the opioid and/or opiate for a period of at least about 1 day (d), 2 d, 3 d, 4 d, 5 d, 6 d, 1 week (w), 2 w, 3 w, 4 w or longer. This administration may be in a clinical setting, for example as described above for IOWS. The opioid and/or opiate may be any that has the potential to induce physical and/or somatic withdrawal in the subject. These opioids and opiates include oxycodone, morphine, buprenorphine, codeine, fentanyl, opium, methadone, heroin, hydrocodone, hydromorphone, oxymorphone, meperidine, tramadol, propoxyphene, diphenoxylate, loperamide, nalbuphine, butorphanol, pentazocine, carfentanil and other fentanyl analogues, and combinations thereof. In some embodiments, these opioids and/or opiates may be referred to as opioid agonists, as these compounds are all agonists or partial agonists of one or more opioid receptors.

In some embodiments, administration of the compound of Formula (I) may be initiated after cessation of administration of an opioid and/or opiate. In some embodiments, administration of a compound of Formula (I) or a pharmaceutically acceptable salt and/or prodrug thereof is initiated within at least about 30 minutes (m), 45 m, 1 hour (h), 1.5 h, 2 h, 5 h, 10 h, 12 h, 18 h or 24 h of the last dose of the opioid and/or opiate.

In some embodiments, administration of the compound of Formula (I) may be initiated prior to cessation of administration of the opioid and/or opiate. In some embodiments, administration of the first dose of the compound of Formula (I) occurs simultaneously with the last dose of opioid and/or opiate. In some embodiments, administration of the first dose of the compound of Formula (I) occurs at least about 1, 2, 3, 4, 5, 6, 7, 8, 9, 10, 11, 12, 13 or 14 days or longer prior to the last dose of the opioid and/or opiate.

In some embodiments, administration of the compound of Formula (I) may be initiated prior to administration of the opioid and/or opiate. Such methods may be useful, for example, prior to a surgical procedure where opioids are likely to be required during the procedure or during recovery.

It will be appreciated that when a compound of Formula (I) is administered prior to cessation or reduction of opioid and/or opiate use (including where administration of the compound of Formula (I) precedes opioid and/or opiate administration), opioid withdrawal and/or a symptom associated with the opioid withdrawal may be prevented in the subject. Accordingly, in some embodiments, the methods of the invention are methods of preventing opioid withdrawal and/or a symptom associated with the opioid withdrawal, comprising administering to a subject at risk of opioid withdrawal an effective amount of a compound of Formula (I) or a pharmaceutically acceptable salt and/or prodrug thereof to thereby prevent opioid withdrawal and/or a symptom associated with the opioid withdrawal.

In some embodiments, the method comprises tapering a dose of the opioid and/or opiate. Tapering comprises reducing the dose of the opioid and/or opiate in a step-wise manner over a period of time. Suitable tapering regimens will depend on the particular opioid and/or opiate and other factors understood by the person skilled in the art.

In some embodiments, administering the compound of Formula (I) is initiated prior to commencement of opioid/opiate dose tapering.

In some embodiments, administering the compound of Formula (I) is initiated concurrently with commencement of opioid/opiate dose tapering. In these embodiments, the compound of formula (I) may be administered within about 15 m, 30 m, 60 m or 120 m of the first dose of the tapering regimen of the opioid and/or opiate (eg the last highest dose of the opioid and/or opiate).

In some embodiments, administering the compound of Formula (I) is initiated during opioid/opiate dose tapering, for example when the opioid and/or opiate dosage has been reduced by about 5%, 10%, 15%, 20%, 25%, 30%, 40%, 50%, 60%, 70%, 75%, 80%, 90% or 95%.

In some embodiments, administering the compound of Formula (I) is initiated following conclusion of opioid/opiate dose tapering. In some embodiments, administration of a compound of Formula (I) or a pharmaceutically acceptable salt and/or prodrug thereof is initiated within at least about 30 minutes (mins), 45 mins, 1 hour (h), 1.5 h, 2 h, 5 h, 10 h, 12 h, 18 h or 24 h of the last dose of the opioid.

In some embodiments, administration of the compound of Formula (I) may be maintained for at least about 1 week (w), 2 w, 3 w, 4 w, 5 w, 6 w, 7 w, 8 w, 9 w, 10 w, 11 w, 12 w or longer. The length of time that the administration is maintained will depend on the opioid and/or opiate causing the withdrawal symptoms, the individual subject and the length of any co-administration period. In some embodiments, administration of the compound of Formula (I) may be stopped and restarted if the subject experiences the onset of a symptom of opioid withdrawal after the initial treatment period.

In some embodiments, the opioid withdrawal is neonatal opioid withdrawal. In these embodiments, exposure to the opioid and/or opiate occurs in utero.

In some embodiments, opioid withdrawal may be induced by administration of an opioid antagonist or partial agonist. Accordingly, the methods may comprise administration of an opioid antagonist or partial agonist. Typically, the opioid antagonists include naloxone and naltrexone and the opioid partial agonist includes buprenorphine. In these methods, the compound of Formula (I) may be administered concurrently, before or after administration of the opioid antagonist or partial agonist. Preferably, the compound of Formula (I) is administered concurrently with the opioid antagonist or partial agonist as administration of the opioid antagonist or partial agonist may induce opioid withdrawal.

Opioid antagonists may be used to treat opioid overdose. In some embodiments, the methods of the invention may be used in methods of treating opioid overdose. Thus, also described herein are methods of treating opioid overdose, comprising administering to a subject in need thereof an effective amount of a compound of Formula (I) or a pharmaceutically acceptable salt and/or solvate thereof in combination with an effective amount of an opioid antagonist. The compound of Formula (I) or a pharmaceutically acceptable salt and/or solvate thereof may be administered by the same or different route to the opioid antagonist.

Also described herein are methods of treating pain, comprising administering an effective amount of an opioid and/or opiate and an effective amount of a compound of Formula (I) or a pharmaceutically acceptable salt or prodrug thereof. The pain may be any pain for which opioid and/or opiate therapy may be effective. Administration of the compound of Formula (I) or pharmaceutically acceptable salt and/or prodrug thereof in these methods is intended to prevent, or reduce the severity of, opioid withdrawal and/or a symptom associated with the opioid withdrawal. Accordingly, the administration of the compound of Formula (I) in these methods may be according to its administration in any of the methods of treating opioid withdrawal and/or a symptom associated with the opioid withdrawal described herein.

### Compounds

The methods of the invention comprise administering a compound of Formula (I)

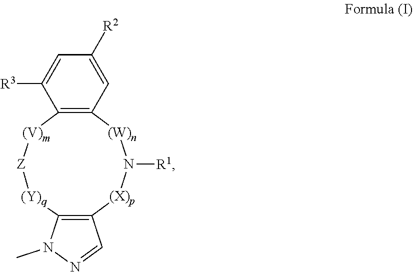

wherein:

V is NH, CH₂ or a direct bond;

W is NH, CH₂ or a direct bond

X is NH, CH₂ or a direct bond

Y is NH, CH₂ or a direct bond

Z is selected from: NH, O, S, S(O), SO₂ or a direct bond;

R¹ is selected from H or C(O)R⁴;

R² is selected from: H, OH, halogen, an optionally substituted C₁₋₅ alkyl or an optionally substituted OC₁₋₅ alkyl;

R³ is selected from: H, OH, halogen, an optionally substituted C₁₋₅ alkyl or an optionally substituted OC₁₋₅ alkyl;

R⁴ is an optionally substituted C₁₋₅ alkyl;

m is 0 or 1;

n is 0 or 1;

p is 0 or 1; and

q is 0 or 1.

In some embodiments, compounds of formula (I) may be provided as compounds of Formula (Ia),

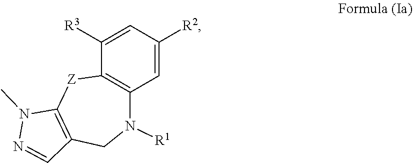

wherein:

Z is selected from: NH, O, S, S(O) or SO₂;

R¹ is selected from H or C(O)R⁴;

R² is selected from: H, OH, halogen, an optionally substituted C₁₋₅ alkyl or an optionally substituted OC₁₋₅ alkyl;

R³ is selected from: H, OH, halogen, an optionally substituted C₁₋₅ alkyl or an optionally substituted OC₁₋₅ alkyl; and

R⁴ is an optionally substituted C₁₋₅ alkyl.

In some embodiments, compounds of formula (I) may be provided as compounds of Formula (Ib),

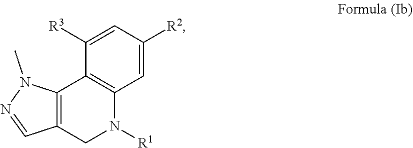

wherein:

R¹ is selected from H or C(O)R⁴;

R² is selected from: H, OH, halogen, an optionally substituted C₁₋₅ alkyl or an optionally substituted OC₁₋₅ alkyl;

R³ is selected from: H, OH, halogen, an optionally substituted C₁₋₅ alkyl or an optionally substituted OC₁₋₅ alkyl; and

R⁴ is an optionally substituted C₁₋₅ alkyl.

In some embodiments, compounds of formula (I) may be provided as compounds of Formula (Ic) or salts or prodrugs thereof,

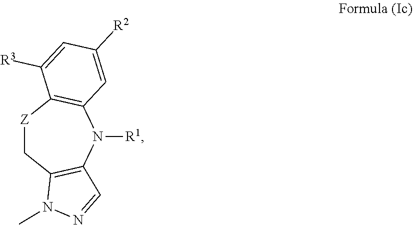

wherein:

Z is selected from: NH, O, S, S(O) or SO₂;

R¹ is selected from H or C(O)R⁴;

R² is selected from: H, OH, halogen, an optionally substituted C₁₋₅ alkyl or an optionally substituted OC₁₋₅ alkyl;

R³ is selected from: H, OH, halogen, an optionally substituted C₁₋₅ alkyl or an optionally substituted OC₁₋₅ alkyl; and

R⁴ is an optionally substituted C₁₋₅ alkyl.

In some embodiments, compounds of formula (I) may be provided as compounds of Formula (Id)

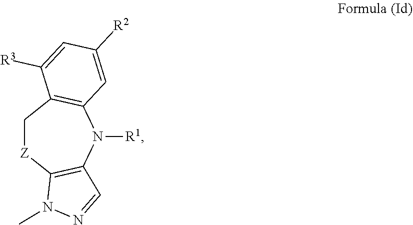

wherein:

Z is selected from: NH, O, S, S(O) or SO₂;

R¹ is selected from H or C(O)R⁴;

R² is selected from: H, OH, halogen, an optionally substituted C₁₋₅ alkyl or an optionally substituted OC₁₋₅ alkyl;

R³ is selected from: H, OH, halogen, an optionally substituted C₁₋₅ alkyl or an optionally substituted OC₁₋₅ alkyl; and

R⁴ is an optionally substituted C₁₋₅ alkyl.

In some embodiments, compounds of formula (I) may be provided as compounds of Formula (Ie)

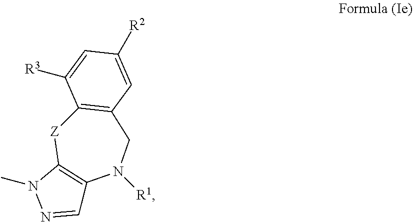

wherein:

Z is selected from: NH, O, S, S(O) or SO₂;

R¹ is selected from H or C(O)R⁴;

R² is selected from: H, OH, halogen, an optionally substituted C₁₋₅ alkyl or an optionally substituted OC₁₋₅ alkyl;

R³ is selected from: H, OH, halogen, an optionally substituted C₁₋₅ alkyl or an optionally substituted OC₁₋₅ alkyl; and

R⁴ is an optionally substituted C₁₋₅ alkyl.

In some embodiments, compounds of formula (I) may be provided as compounds of Formula (If)

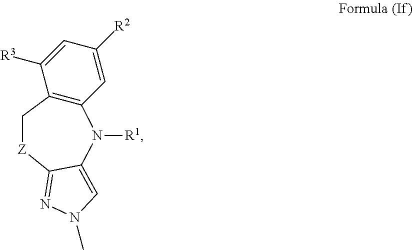

wherein:

Z is selected from: NH, O, S, S(O) or SO₂;

R¹ is selected from H or C(O)R⁴;

R² is selected from: H, OH, halogen, an optionally substituted C₁₋₅ alkyl or an optionally substituted OC₁₋₅ alkyl;

R³ is selected from: H, OH, halogen, an optionally substituted C₁₋₅ alkyl or an optionally substituted OC₁₋₅ alkyl; and

R⁴ is an optionally substituted C₁₋₅ alkyl.

In some embodiments the compound of Formula (I) is a compound of Formula (Ia), or a salt or prodrug thereof.

In some embodiments the compound of Formula (I) is a compound of Formula (Ib), or a salt or prodrug thereof.

In some embodiments the compound of Formula (I) is a compound of Formula (Ic), or a salt or prodrug thereof.

In some embodiments the compound of Formula (I) is a compound of Formula (Id), or a salt or prodrug thereof.

In some embodiments the compound of Formula (I) is a compound of Formula (Ie), or a salt or prodrug thereof.

In some embodiments the compound of Formula (I) is a compound of Formula (If), or a salt or prodrug thereof.

In some embodiments V is NH.

In some embodiments V is CH₂

In some embodiments V is a direct bond;

In some embodiments W is NH.

In some embodiments W is CH₂.

In some embodiments W is a direct bond.

In some embodiments X is NH.

In some embodiments X is CH₂.

In some embodiments X is a direct bond

In some embodiments Y is NH.

In some embodiments Y is CH₂.

In some embodiments Y is a direct bond

In some embodiments Z is NH.

In some embodiments Z is O.

In some embodiments Z is S.

In some embodiments Z is S(O).

In some embodiments Z is SO₂.

In some embodiments Z is a direct bond.

In some embodiments R¹ is hydrogen.

In some embodiments R¹ is C(O)R⁴. For example, R⁴ may be an optionally substituted C₁-5 alkyl selected from: methyl, ethyl, propyl, isopropyl, n-butyl, sec-butyl, tert-butyl, n-pentyl, neopentyl, iso-pentyl and tert-pentyl groups. In some embodiments R⁴ is an optionally substituted methyl.

In some embodiments R² is hydrogen.

In some embodiments R² is a hydroxyl group.

In some embodiments R² is a halogen. For example, in some embodiments R² is fluorine. In another embodiment R² is chlorine.

In some embodiments R² is an optionally substituted C₁₋₅ alkyl. For example, R² may be an optionally substituted C₁-5 alkyl selected from: methyl, ethyl, propyl, isopropyl, n-butyl, sec-butyl, tert-butyl, n-pentyl, neopentyl, iso-pentyl and tert-pentyl. In some embodiments R² is be an optionally substituted methyl.

In some embodiments R² is an optionally substituted OC₁₋₅ alkyl. For example, R² may be an optionally substituted OC₁₋₅ alkyl selected from: methoxy, ethoxy, propyloxy, isopropyloxy, n-butyloxy, sec-butyloxy, tert-butyloxy, n-pentyloxy, neopentyloxy, iso-pentyloxy and tert-pentyloxy groups. In some embodiments R² is an optionally substituted methoxy group.

In some embodiments R³ is hydrogen.

In some embodiments R³ is a hydroxyl group.

In some embodiments R³ is a halogen. For example, in some embodiments R³ is fluorine. In another embodiment R³ is chlorine.

In some embodiments R³ is an optionally substituted C₁₋₅ alkyl. For example, R³ may be an optionally substituted C₁₋₅ alkyl selected from: methyl, ethyl, propyl, isopropyl, n-butyl, sec-butyl, tert-butyl, n-pentyl, neopentyl, iso-pentyl and tert-pentyl. In some embodiments R³ is be an optionally substituted methyl.

In some embodiments R³ is an optionally substituted OC₁₋₅ alkyl. For example, R³ may be an optionally substituted OC₁₋₅ alkyl selected from: methoxy, ethoxy, propyloxy, isopropyloxy, n-butyloxy, sec-butyloxy, tert-butyloxy, n-pentyloxy, neopentyloxy, iso-pentyloxy and tert-pentyloxy groups. In some embodiments R³ is an optionally substituted methoxy group.

In some embodiments the compound of Formula (I) is selected from:

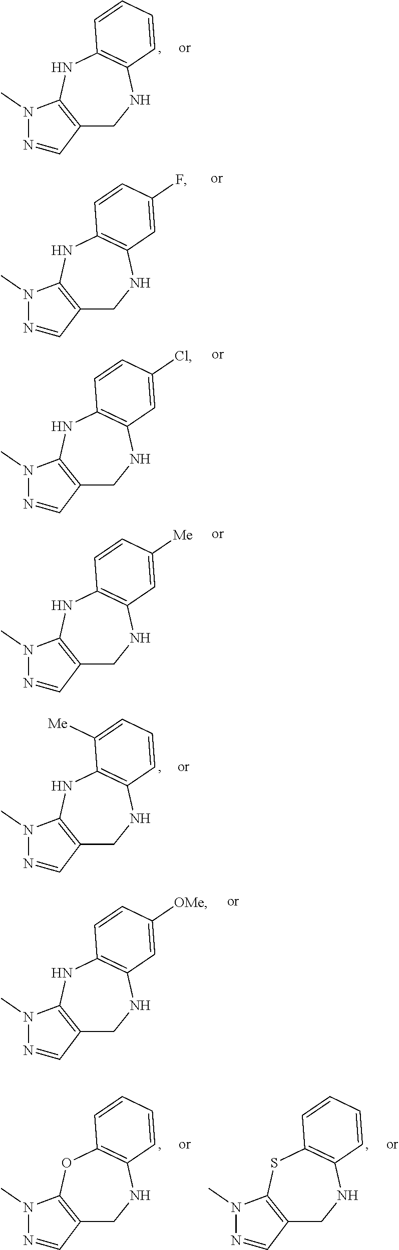

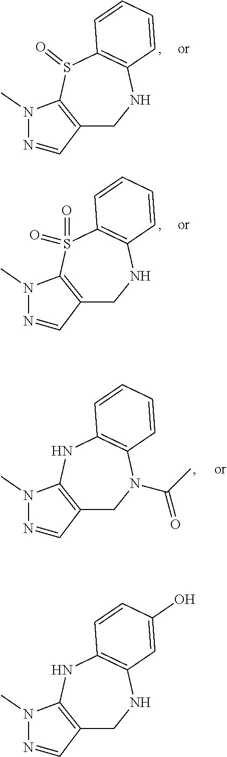

or a salt or prodrug thereof.

In another embodiment, the compound of Formula (I) is selected from:

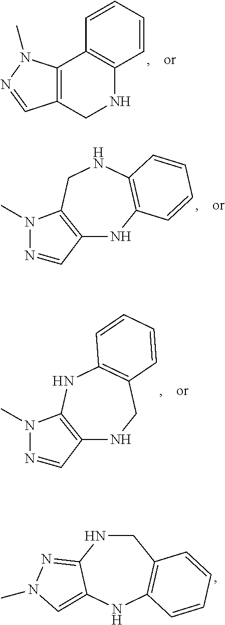

or a salt or prodrug thereof.

In another embodiment compound of Formula (I) is selected from:

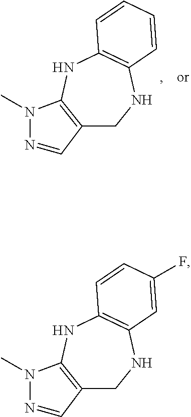 [SMILES: Cn1ncc2c1Nc1ccccc1NC2 ; Cn1ncc2c1Nc1ccc(F)cc1NC2]

or a salt or prodrug thereof. For example, in some embodiments the compound of Formula (I) is:

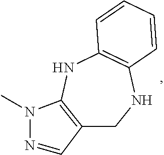

or a salt or prodrug thereof.

The methods may comprise administering the compound of Formula (I) in any pharmaceutically acceptable form. In some embodiments, the compound of Formula (I) is provided in the form of a pharmaceutically acceptable salt, solvate, N-oxide, polymorph, tautomer or prodrug thereof, or a combination of these forms in any ratio.

In some embodiments the compound of Formula (I) is a salt, for example a pharmaceutically acceptable salt.

Suitable pharmaceutically acceptable salts include, but are not limited to: salts of pharmaceutically acceptable inorganic acids such as: hydrochloric, sulfuric, phosphoric, nitric, carbonic, boric, sulfamic, and hydrobromic acids; or salts of pharmaceutically acceptable organic acids such as acetic, propionic, butyric, tartaric, maleic, hydroxymaleic, fumaric, isethionic, malic, citric, lactic, mucic, gluconic, benzoic, succinic, oxalic, phenylacetic, methanesulfonic, toluenesulfonic, benzenesulfonic, salicylic, sulfanilic, aspartic, glutamic, edetic, stearic, palmitic, oleic, lauric, pantothenic, tannic, ascorbic and valeric acids.

Base salts include, but are not limited to: those formed with pharmaceutically acceptable cations, such as: sodium, potassium, lithium, calcium, magnesium, zinc, ammonium and alkylammonium; salts formed from triethylamine; alkoxyammonium salts such as those formed with ethanolamine; and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1^{st} edition, 2002, Wiley-VCH.

Basic nitrogen-containing groups in Formula (I) (or basic nitrogen-containing groups in a compound of Formula (Ia), Formula (Ib), Formula (Ic), Formula (Id), Formula (Ie), or Formula (If)), may be quarternised with such agents as C₁₋₆ alkyl halide, such as methyl, ethyl, propyl, and butyl chlorides, bromides and iodides; dialkyl sulfates like dimethyl and diethyl sulfate; and others known in the art.

In some embodiments the compound of Formula (I) is a salt of a compound selected from:

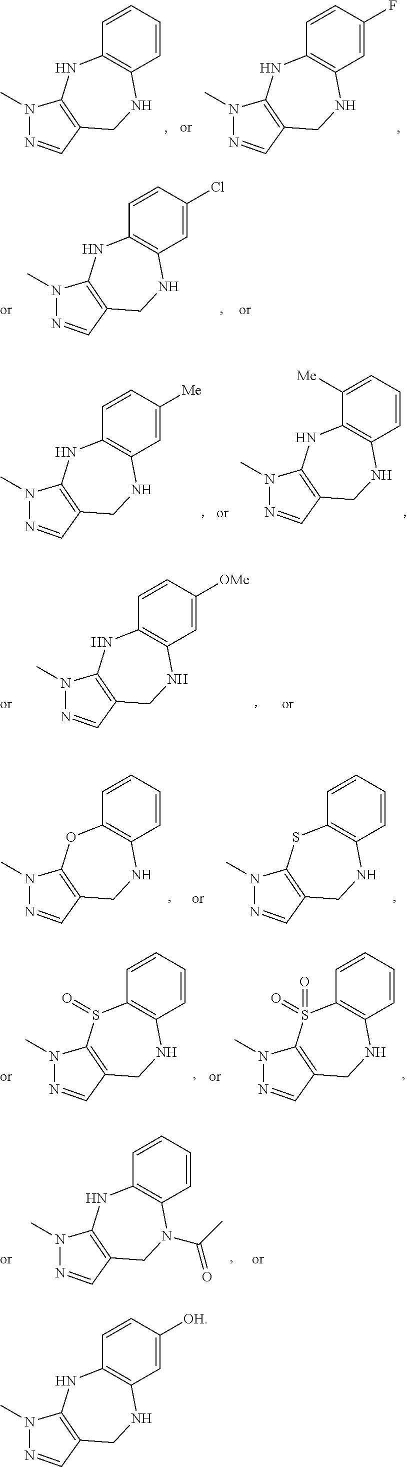

In another embodiment, the compound of Formula (I) is a salt of a compound selected from:

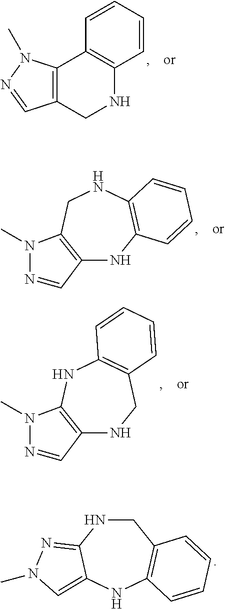

In another embodiment, the compound of Formula (I) is a salt of a compound selected from:

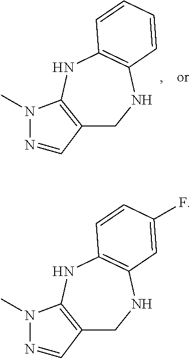 [SMILES: Cn1ncc2c1Nc1ccccc1NC2 ; Cn1ncc2c1Nc1ccc(F)cc1NC2]

For example, in some embodiments, the salt of a compound of Formula (I) is a salt of

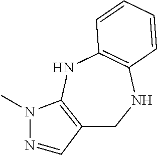

Whilst, in yet another embodiment, the salt of a compound of Formula (I) is a salt of

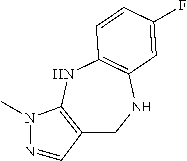 [SMILES: Cn1ncc2c1Nc1ccc(F)cc1NC2]

In some embodiments the compound of Formula (I) is a hydrochloride salt.

In some embodiments, the hydrochloride salt is:

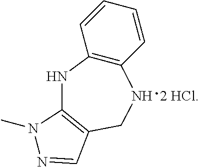

In some embodiments the compound of Formula (I) is a phosphoric acid addition salt.

Prodrugs include compounds wherein an amino acid residue, or a polypeptide chain of two or more (e.g., two, three or four) amino acid residues which are covalently joined to free amino, hydroxy and carboxylic acid groups of compounds of Formula (I). The amino acid residues include the 20 naturally occurring amino acids commonly designated by three letter symbols and also include: 4-hydroxyproline, hydroxylysine, demosine, isodemosine, 3-methylhistidine, norvaline, beta-alanine, gamma-aminobutyric acid, citrulline, homocysteine, homoserine, ornithine and methionine sulfone. Prodrugs also include compounds wherein carbonates, carbamates, amides and alkyl esters, which may be covalently bonded to the above substituents of Formula (I) through the carbonyl carbon prodrug side chain. Prodrugs also include phosphate derivatives of compounds of Formula (I) (such as acids, salts of acids, or esters) joined through a phosphorus-oxygen bond to a free hydroxyl of compounds of Formula (I). It will be understood that the invention disclosed and defined in this specification extends to all alternative combinations of two or more of the individual features mentioned or evident from the text or drawings. All of these different combinations constitute various alternative aspects of the invention.

The compounds of Formula (I) or salts, tautomers, N-oxides, polymorphs or prodrugs thereof may be provided in the form of solvates. Solvates contain either stoichiometric or non-stoichiometric amounts of a solvent, and may be formed during the process of crystallization with pharmaceutically acceptable solvents such as water, alcohols such as methanol, ethanol or isopropyl alcohol, DMSO, acetonitrile, dimethyl formamide (DMF) and the like with the solvate forming part of the crystal lattice by either non-covalent binding or by occupying a hole in the crystal lattice. Hydrates are formed when the solvent is water, alcoholates are formed when the solvent is alcohol. Solvates of the compounds of the present invention can be conveniently prepared or formed during the processes described herein. In general, the solvated forms are considered equivalent to the unsolvated forms for the purposes of the compounds and methods provided herein.

The compound of Formula (I) or salts, tautomers, N-oxides, solvates and/or prodrugs thereof that form crystalline solids may demonstrate polymorphism. All polymorphic forms of the compounds, salts, tautomers, N-oxides, solvates and/or prodrugs may be used in the methods of the invention.

The compound of Formula (I) may demonstrate tautomerism. Tautomers are two interchangeable forms of a molecule that typically exist within an equilibrium. Any tautomers of the compounds of Formula (I) may be used in the methods of the invention.

Methods for making compounds of Formula (I) are described in PCT/AU2016/050588.

### Administration

The compounds of Formula (I), or a pharmaceutically acceptable salt or prodrug thereof, as defined herein, may be administered by any suitable means, for example, orally, rectally, nasally, vaginally, topically (including buccal and sub-lingual), parenterally, such as by subcutaneous, intraperitoneal, intravenous, intramuscular, or intracisternal injection, inhalation, insufflation, infusion or implantation techniques (e.g., as sterile injectable aqueous or non-aqueous solutions or suspensions).

The compounds of the invention may be provided as pharmaceutical compositions including those for oral, rectal, nasal, topical (including buccal and sub-lingual), parenteral administration (including intramuscular, intraperitoneal, sub-cutaneous and intravenous), or in a form suitable for administration by inhalation or insufflation. The compounds of Formula (I), or a pharmaceutically acceptable salt or prodrug thereof, together with a conventional adjuvant, carrier or diluent, may thus be placed into the form of pharmaceutical compositions and unit dosages thereof, and in such form may be employed as solids, such as tablets or filled capsules, or liquids as solutions, suspensions, emulsions, elixirs or capsules filled with the same, all for oral use, or in the form of sterile injectable solutions for parenteral (including subcutaneous) use.

The pharmaceutical compositions for the administration of the compounds of this invention may conveniently be presented in dosage unit form and may be prepared by any of the methods well known in the art of pharmacy (See, for example, Remington: The Science and Practice of Pharmacy, 21st Ed., 2005, Lippincott Williams & Wilkins). All methods include the step of bringing the active ingredient, for example a compound defined by Formula (I), or a pharmaceutically acceptable salt or prodrug thereof, into association with the carrier which constitutes one or more accessory ingredients. In general, the pharmaceutical compositions are prepared by uniformly and intimately bringing the active ingredient, for example a compound defined by Formula (I), or a pharmaceutically acceptable salt or prodrug thereof, into association with a liquid carrier or a finely divided solid carrier or both, and then, if necessary, shaping the product into the desired formulation. In the pharmaceutical composition the active object compound is included in an amount sufficient to produce the desired effect. In some embodiments, the method of the invention comprises administering a pharmaceutical comprising a compound of Formula (I) or a pharmaceutically acceptable salt or solvate thereof and a pharmaceutically acceptable carrier, diluent and/or excipient.

Compounds of Formula (I), or a pharmaceutically acceptable salt or prodrug thereof, may be administered in a dose of about 0.001, 0.005, 0.01, 0.05, 0.1, 0.15, 0.2, 0.5, 1, 2, 3, 5, 10, 15, 20, 25 or 30 mg/kg of the body weight of the subject. In some embodiments, the dose may be from any of these amounts to any other amount, such as from about 0.001 mg/kg to about 30 mg/kg, about 0.2 mg/kg to about 30 mg/kg or about 0.2 mg/kg to about 10 mg/kg. It will be understood, however, that the specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Compounds of Formula (I), or a pharmaceutically acceptable salt or prodrug thereof, may be provided in an “effective amount”, for example when an appropriate compound is added to a pharmaceutical composition. “Effective amount” is taken to mean an amount of a compound that will elicit a desired biological or medical response of a tissue, system, animal or human that is being sought by the researcher, veterinarian, medical doctor or other clinician administering the compound of a composition including the compound. In some embodiments, the effective amount may be a “therapeutically effective amount” wherein the amount of the object active compound is effective to treat the condition and/or symptom thereof that has manifested in the subject. In other embodiments, the effective amount may be a “prophylactically effective amount” wherein the amount of the object active compound is sufficient to prophylactically treat and/or prevent the onset of the condition and/or a symptom associated with the opioid withdrawal or, if a symptom emerges, cause the severity of the condition and/or symptom thereof to be at a reduced level compared to the average severity of the condition and/or symptom thereof in a population of subjects not having received treatment with the compound of Formula (I) and/or a pharmaceutically acceptable salt and/or prodrug thereof.

An “effective amount” is that amount of a compound of Formula (I), or a pharmaceutically acceptable salt or prodrug thereof, provided herein, the administration of which to a subject, either in a single dose or as part of a series, is effective to manage and/or prevent one or more symptoms of opioid withdrawal. An amount is effective, for example, when its administration results in one or more of cessation of a symptom of opioid withdrawal, alleviation of severity of a symptom of opioid withdrawal, reduction in duration the subject experiences the withdrawal symptom, prevention of the severity of the symptom that emerges, prevention of onset of a symptom of opioid withdrawal and/or prevention of worsening of a symptom of opioid withdrawal.

The “effective amount” will be dependent on a number of factors, including the efficacy of the particular compound, physical condition of the subject to be treated, the severity of opioid withdrawal symptoms, the formulation of the compound, and/or a professional assessment of the medical situation. The subject's weight and age may also be a factor for the person skilled in the art when determining the amount of compound that the subject should receive.

In some embodiments, the methods of the invention treat a symptom of opioid withdrawal. The symptoms of opioid withdrawal include psychological, physical and/or somatic symptoms.

Physical and somatic symptoms of opioid withdrawal include tremors, shaking, hot or cold flashes, goosebumps, sweating, rapid breathing, elevated heart rate, elevated blood pressure, body aches, vomiting, diarrhea and fever. In some embodiments, methods treat a physical and/or somatic symptom of opioid withdrawal. In some embodiments, the physical and/or somatic symptoms are selected from tremors and shaking.

Psychological symptoms of opioid withdrawal include dysphoria, anxiety, restlessness, irritability, insomnia, yawning, hallucinations, hyperalgesia, hyperkatifiteia, and anorexia. It is believed that although these symptoms are not physical/somatic, they are symptoms of opioid withdrawal and stem from the physiological changes resulting from cessation or reduction of opioid dosing and/or induced by opioid antagonist administration. In some embodiments, the methods treat dysphoria.

Symptoms of opioid withdrawal include dysphoria, anxiety, restlessness, irritability, insomnia, yawning, hallucinations, tremors, shaking, hot or cold flashes, goosebumps, sneezing, sweating, rapid breathing, elevated heart rate, elevated blood pressure, pupillary dilation, piloerection, head aches, body aches, muscle cramps, muscle aches, bone aches, joint aches, hyperalgesia, hyperkatifiteia, watery discharge from eyes and nose (lacrimation and rhinorrhea), nausea, vomiting, diarrhea, abdominal pain, anorexia and fever. As noted above, one of the diagnostic tools developed regarding opioid withdrawal is the DSM-5. The DSM-5 specifies that for a subject to be diagnosed with opioid withdrawal, 3 of the following 9 symptoms must develop within minutes to several days of either cessation (or reduction) of opioid exposure, or the administration of an opioid antagonist or partial agonist. The DSM-5 symptoms are (1) dysphoric mood, (2) nausea, (3) muscle aches, (4) lacrimation or rhinorrhea, (5) pupillary dilation, piloerection or sweating, (6) diarrhea, (7) yawning, (8) fever and (9) insomnia. Accordingly, in some embodiments, the subject experiences at least 1, 2, 3, 4, 5, 6, 7, 8 or 9 of these DSM-5 symptoms and preferably administration of the compound of Formula (I) treats at least one of the symptoms experienced by the subject.

The severity of withdrawal symptoms will depend on the opioid causing the dependence, the dose and length of treatment or abuse, how rapidly opioid use is discontinued and the characteristics of the subject including age, sex, weight etc.

Accordingly, in some embodiments, the methods treat an opioid withdrawal symptom selected from the group consisting of tremors, shaking, hot or cold flashes, goosebumps, sweating, rapid breathing, elevated heart rate, elevated blood pressure, body aches, vomiting, diarrhea, fever, dysphoria, anxiety, restlessness, irritability, insomnia, yawning, hallucinations, hyperalgesia, hyperkatifiteia, and anorexia, or a combination thereof.

The phrases “administration of” and or “administering a” compound should be understood to mean providing the object active compound (for example a compound of Formula (I) (or a compound of Formula (Ia), Formula (Ib), Formula (Ic), Formula (Id) or Formula (Ie)), or a pharmaceutically acceptable salt or prodrug thereof; an opioid and/or opiate or an opioid antagonist or partial agonist) to a subject in need thereof.

As provided herein, beneficial or desired clinical results from the disclosed compound of Formula (I), or a pharmaceutically acceptable salt or prodrug thereof, include, without limitation, cessation of a symptom of opioid withdrawal, alleviation of severity of a symptom of opioid withdrawal, prevention of onset of a symptom of opioid withdrawal, and/or managing a symptom of opioid withdrawal for example preventing worsening of severity of a symptom of opioid withdrawal or causing the symptom to reduce in severity or cease within a shorter than expected time. Either therapeutic or preventative measures may be achieved. Those in need of treatment include those already experiencing opioid withdrawal as well as those in which opioid withdrawal is to be prevented. By treatment is meant inhibiting or reducing an increase in opioid withdrawal symptoms when compared to the absence of treatment, and is not necessarily meant to imply complete cessation of the relevant condition.

Thus, generally, the term “treatment” (and variations thereof including “treating”) means affecting a subject, tissue or cell to obtain a desired pharmacological and/or physiological effect and includes: (a) cessation of a symptom of opioid withdrawal, (b) alleviation of severity of a symptom of opioid withdrawal, (c) reduction in duration the subject experiences the withdrawal symptom, (d) prevention of the severity of the symptom that emerges, (e) prevention of onset of a symptom of opioid withdrawal and/or (f) prevention of worsening of a symptom of opioid withdrawal. In some embodiments, the symptom to be treated is a physical and/or somatic symptom of opioid withdrawal. References to managing opioid withdrawal in the context of the methods disclosed herein are intended to encompass treating opioid withdrawal by affecting any of these desired pharmacological and/or physiological effects to within a subject's tolerance for opioid withdrawal symptoms.

In another aspect, also provided is a method of treating and/or managing opioid withdrawal, comprising administering to a subject in need thereof an effective amount of a compound of Formula (I) or a pharmaceutically acceptable salt or prodrug thereof.

In another aspect, also provided is a method of treating and/or controlling opioid withdrawal symptoms, comprising administering to a subject in need thereof an effective amount of a compound of Formula (I) or a pharmaceutically acceptable salt or prodrug thereof.

In another aspect, also provided is use of a compound of Formula (I) or a pharmaceutically acceptable salt or prodrug thereof in the preparation of a medicament for:
treating, managing and/or controlling a symptom associated with opioid withdrawal; and/or
treating and/or managing opioid withdrawal.

In another aspect, also provided is use of a compound of Formula (I) or a pharmaceutically acceptable salt or prodrug thereof for:
treating, managing and/or controlling a symptom associated with opioid withdrawal; and/or
treating and/or managing opioid withdrawal.

In another aspect, also provided is a compound of Formula (I) or a pharmaceutically acceptable salt or prodrug thereof for use in:
treating, managing and/or controlling a symptom associated with opioid withdrawal; and/or
treating and/or managing opioid withdrawal.

In another aspect, also provided is a composition comprising a compound of Formula (I) or a pharmaceutically acceptable salt or prodrug thereof for use in:
treating, managing and/or controlling a physiological symptom associated with opioid withdrawal;
treating and/or managing opioid withdrawal.

Any embodiment described herein in relation to the administration step or a compound of the invention may be employed in these compounds, compositions, uses and/or methods.

Kits

Also provided is a kit of parts, comprising in separate parts:
a compound of Formula (I) or a pharmaceutically acceptable salt or prodrug thereof; and
instructions for its use in any of the methods of the invention.

Also provided is a kit of parts comprising in separate parts:
a compound of Formula (I) or a pharmaceutically acceptable salt or prodrug thereof; and
an opioid and/or opiate.

In some embodiments, the part comprising the opioid and/or opiate comprises the opioid and/or opiate in a plurality of unit dose form, such as those suitable for a tapering regimen.

Also provided is a kit of parts comprising in separate parts:
a compound of Formula (I) or a pharmaceutically acceptable salt or prodrug thereof; and
an opioid antagonist and/or partial agonist.

In any of the kits disclosed herein, the compound of Formula (I) or a pharmaceutically acceptable salt or prodrug thereof and/or the opioid and/or opiate and/or the opioid antagonist and/or partial agonist may be formulated as a pharmaceutical composition together with a pharmaceutically acceptable carrier, diluent and/or excipient. The pharmaceutical compositions may be formulated for administration by any route disclosed herein including for oral, rectal, nasal, topical (including buccal and sub-lingual), parenteral administration (including intramuscular, intraperitoneal, sub-cutaneous and intravenous), or in a form suitable for administration by inhalation or insufflation.

### Compositions

In another aspect, the invention provides a pharmaceutical composition comprising an effective amount of a compound of Formula (I) or a pharmaceutically acceptable salt and/or prodrug thereof and an opioid and/or opiate.

Any compound of Formula (I) or a salt and/or prodrug thereof described herein may be included in these compositions.

Any opioid and/or opiate described herein may be included in these compositions.

In some embodiments, the opioid and/or opiate is provided in an effective amount, any may be any amount that presents a risk of opioid withdrawal to the subject. In other embodiments, the opioid and/or opiate is provided in an amount suitable for administration according to a tapering regimen.

The pharmaceutical compositions comprising the compound of Formula (I) or a salt and/or prodrug thereof and an opioid and/or opiate may be prepared in any of the forms described herein for administering the compound of Formula (I), including those for oral, rectal, nasal, topical (including buccal and sub-lingual), parenteral administration (including intramuscular, intraperitoneal, sub-cutaneous and intravenous), or in a form suitable for administration by inhalation or insufflation.

In another aspect, the invention provides a pharmaceutical composition comprising an effective amount of a compound of Formula (I) or a pharmaceutically acceptable salt and/or prodrug thereof and an effective amount of an opioid antagonist and/or partial agonist.

Any compound of Formula (I) or a salt and/or prodrug thereof described herein may be included in these compositions.

Any opioid antagonist and/or partial agonist described herein may be included in these compositions.

The pharmaceutical compositions comprising the compound of Formula (I) or a salt and/or prodrug thereof and an opioid antagonist and/or partial agonist may be prepared in any of the forms described herein for administering the compound of Formula (I), including those for oral, rectal, nasal, topical (including buccal and sub-lingual), parenteral administration (including intramuscular, intraperitoneal, sub-cutaneous and intravenous), or in a form suitable for administration by inhalation or insufflation.

The pharmaceutical compositions typically further comprise a pharmaceutically acceptable carrier, diluent and/or excipient. Any convention carrier, diluent and/or excipient may be included as known in the art of pharmacy (See, for example, Remington: The Science and Practice of Pharmacy, 21st Ed., 2005, Lippincott Williams & Wilkins).

The pharmaceutical compositions described herein may be used in any of the methods described herein.

### Example 1

This Example describes experiments in a C57BL/6 mouse model of opioid withdrawal (naloxone precipitated withdrawal following oxycodone administration) and the potential of a compound of the invention to treat withdrawal symptoms.

| Abbreviation | Definition |
|---|---|
| i.p. | Intraperitoneal |
| CMPD1 | CMPD1-2HCL |
| | 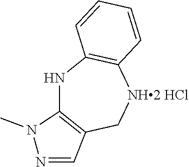 |
| OXCD | Oxycodone |
| OXCD | Mice in the oxycodone condition treated with vehicle i.p. |
| 0CMPD1 | |
| OXCD | Mice in the oxycodone condition treated 10 mg/kg CMPD1- |
| 10CMPD1 | 2HCL i.p. |
| OXCD | Mice in the oxycodone condition treated 2.5 mg/kg CMPD1- |
| 2.5CMPD1 | 2HCL i.p. |
| OXCD | Mice in the oxycodone condition treated 5 mg/kg CMPD1- |
| 5CMPD1 | 2HCL i.p. |
| OXCDVEH | Mice in the oxycodone condition treated with vehicle i.p. |
| VEH | Vehicle |
| VEH | Mice in the vehicle condition treated with vehicle i.p. |
| 0CMPD1 | |
| VEHVEH | Mice in the vehicle condition treated with vehicle i.p. |

Drugs

All drugs used in this study were dissolved in physiological saline and administered at a volume of 10 ml/kg.

### Experiment 1.1: The Effects of CMPD1-2HCL on Locomotor Activity in an Open Field Test

The purpose of this experiment was to demonstrate that CMPD1-2HCL at doses of 5 and 10 mg/kg does not cause potentially confounding effects on locomotor activity. N=18 adult male C57BL/6 mice received either 0, 5 or 10 mg/kg CMPD1-2HCL (i.p.; n=6 per condition). Fifteen minutes after receiving their i.p. injection of CMPD1-2HCL mice were placed individually into a novel 40 (l)×40 (w)×40 (h) cm locomotor testing arena. Sessions were captured by overhead cameras and videos were analysed by using the automated behaviour tracking software CleverSys Topscan (CleverSys, Virginia, USA), which provided the distance travelled by each mouse in each 5 min time bin over the 60 minute locomotor testing session. Data were analysed by SPSS using mixed model ANOVA.

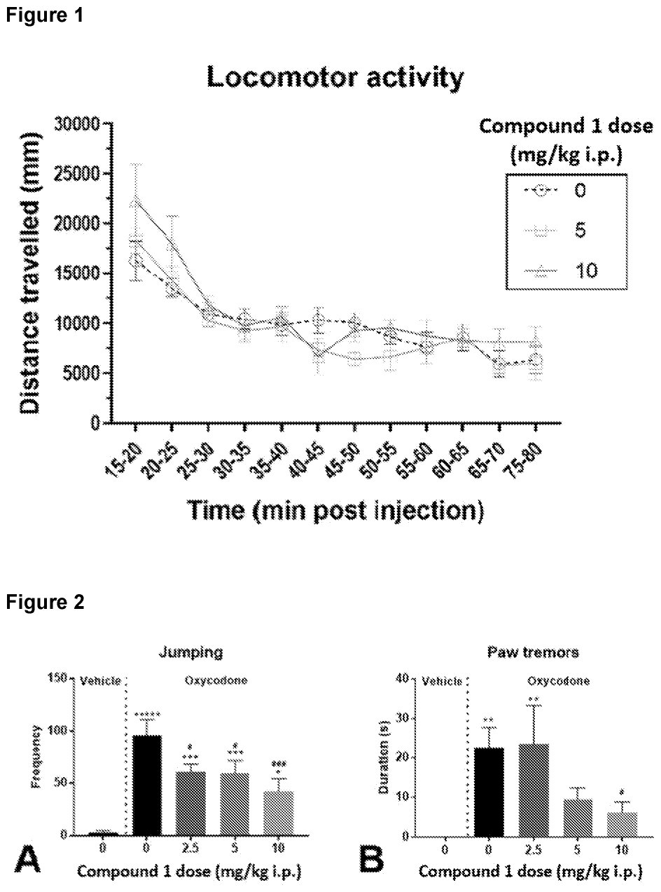

Results of this experiment are shown in Tables 1-3 and FIG. 1.

TABLE 1
Distance (mm) travelled during each time bin for each mouse following 0
mg/kg treatment with CMPD1-2HCL (results of experiment 1 of example 1)

| Time post dose | Mouse 1 | Mouse 2 | Mouse 3 | Mouse 4 | Mouse 5 | Mouse 6 |
|---|---|---|---|---|---|---|
| 15-20 | 15192.33 | 17197.38 | 12075.58 | 25346.9 | 14363.25 | 13217.9 |
| 20-25 | 13796.43 | 13642.5 | 11195.38 | 17036.28 | 14339.16 | 10934.48 |
| 25-30 | 10786.73 | 11503.59 | 6066.88 | 14108.15 | 12381.24 | 10663.14 |
| 30-35 | 9333.18 | 13660.13 | 12654.86 | 10623.36 | 6789.94 | 9632.17 |
| 35-40 | 9769.97 | 11623.57 | 9223.51 | 12816.97 | 5602.56 | 9976.44 |
| 40-45 | 7006.34 | 11044.15 | 5992.5 | 13578.89 | 12776.94 | 11436.02 |
| 45-50 | 9564.41 | 12716.17 | 6100.25 | 9449.26 | 10159 | 12436.17 |
| 50-55 | 6974.93 | 11985.78 | 8306.99 | 8376.14 | 9898.81 | 6509.26 |
| 55-60 | 10056.21 | 9593.22 | 6361.15 | 9089.5 | 9949.84 | 380.8 |
| 60-65 | 9660.61 | 7647.02 | 3862.54 | 8602.72 | 7840.33 | 13892.5 |
| 65-70 | 6195.19 | 11363.52 | 5363.79 | 2432.32 | 3037.29 | 7296.66 |
| 75-80 | 9436.19 | 8779.34 | 3909.8 | 7280.02 | 8391.96 | 431.88 |

TABLE 2
Distance (mm) travelled during each time bin for each mouse following 5
mg/kg treatment with CMPD1-2HCL (results of experiment 1 of example 1)

| Time post dose | Mouse 7 | Mouse 8 | Mouse 9 | Mouse 10 | Mouse 11 | Mouse 12 |
|---|---|---|---|---|---|---|
| 15-20 | 15043.46 | 20271.67 | 23636.81 | 18238.34 | 16051.42 | 16442.58 |
| 20-25 | 20386.17 | 13996.02 | 16449.1 | 11919.46 | 11756.3 | 10974.55 |
| 25-30 | 11089.15 | 11425.88 | 11764.23 | 8462.92 | 10127.95 | 8553.13 |
| 30-35 | 13221.16 | 7779.42 | 11134.18 | 8664.63 | 9823.55 | 4905.54 |
| 35-40 | 15282.34 | 6791.58 | 8208.55 | 11776.57 | 10597.55 | 5280.25 |
| 40-45 | 7068.09 | 9050.59 | 7493.64 | 9121.74 | 5308.25 | 6050.2 |
| 45-50 | 5367.28 | 6712.87 | 5587.1 | 5518.04 | 9993.79 | 5466.54 |
| 50-55 | 945.3 | 7073.77 | 10568.53 | 9463.89 | 5234.55 | 6665.74 |
| 55-60 | 3362.81 | 8088.81 | 11051.63 | 11843.34 | 6386.55 | 4645.85 |
| 60-65 | 6133.83 | 10365.45 | 10059.86 | 11154.54 | 8969.03 | 4893.44 |
| 65-70 | 5268.88 | 4809.55 | 6121.25 | 4100.88 | 9397.98 | 4757.22 |
| 75-80 | 3441.42 | 8422.46 | 11136.92 | 9156.71 | 2402.89 | 1364.63 |

TABLE 3
Distance (mm) travelled during each time bin for each mouse following 5
mg/kg treatment with CMPD1-2HCL (results of experiment 1 of example 1)

| Time post dose | Mouse 13 | Mouse 14 | Mouse 15 | Mouse 16 | Mouse 17 | Mouse 18 |
|---|---|---|---|---|---|---|
| 15-20 | 28970.94 | 12316.3 | 12316.3 | 32680.43 | 27725.23 | 19894.04 |
| 20-25 | 17509.3 | 10864.14 | 10864.14 | 28788.31 | 19945.35 | 19677.12 |
| 25-30 | 10094.38 | 9591.26 | 9591.26 | 14380.51 | 13795.57 | 13450.97 |
| 30-35 | 7690.21 | 6275.9 | 6275.9 | 12268.64 | 12539.86 | 13182.37 |
| 35-40 | 11756.13 | 8046.38 | 8046.38 | 12860.87 | 13271.28 | 10054.85 |
| 40-45 | 7682.41 | 2222.65 | 2222.65 | 11152.11 | 12391.75 | 4683.51 |
| 45-50 | 11290.93 | 6482.48 | 6482.48 | 10743.61 | 12658.5 | 8082.58 |
| 50-55 | Mouse 13 | Mouse 14 | Mouse 15 | Mouse 16 | Mouse 17 | Mouse 18 |
| 55-60 | 28970.94 | 12316.3 | 12316.3 | 32680.43 | 27725.23 | 19894.04 |
| 60-65 | 17509.3 | 10864.14 | 10864.14 | 28788.31 | 19945.35 | 19677.12 |
| 65-70 | 10094.38 | 9591.26 | 9591.26 | 14380.51 | 13795.57 | 13450.97 |
| 75-80 | 7690.21 | 6275.9 | 6275.9 | 12268.64 | 12539.86 | 13182.37 |

The results of this experiment show that CMPD1-2HCL treatment does not significantly impact locomotor activity of the subjects. There was a significant reduction of locomotor activity over time, however, this reduction was consistent across treatment groups (including control) indicating that it was an effect of time and not treatment related. The amount of locomotor activity differed between 5 min bins, averaged across treatment doses [F11,165=29.5, p<0.0001]. There was no significant main effect of CMPD1-2HCL dose [F2,15=0.997, p=0.392] and no significant time by dose interaction effect [F22,165=1.358, p=0.143] for the distance travelled by mice across the test session.

### Experiment 1.2: Assessing the Effects of CMPD1-2HCL on Naloxone Precipitated Oxycodone Withdrawal

Adult male C57BL/6 mice (N=40) were allocated to one of five conditions (n=8 per condition):

(1) Vehicle, 0 mg/kg CMPD1-2HCL;

(2) oxycodone, 0 mg/kg CMPD1-2HCL;

(3) oxycodone, 2.5 mg/kg CMPD1-2HCL;

(4) oxycodone, 5 mg/kg CMPD1-2HCL; or

(5) oxycodone, 10 mg/kg CMPD1-2HCL.

Mice in the oxycodone conditions received i.p. injections of oxycodone for 5 days according to the schedule and doses set out in Table 1. The morning and afternoon doses were separated by 7 h. Mice in the vehicle condition received injections of vehicle saline instead of oxycodone. One-hour-and-forty-five minutes after the morning injection on day 5, mice were administered their i.p. dose of CMPD1-2HCL. Fifteen minutes later they received an i.p. injection of 10 mg/kg naloxone (oxycodone groups) or saline (vehicle group), and proceeded immediately to testing.

TABLE 4
Oxycodone dosing schedule for mice in the oxycodone conditions.

| | Day: | | | | | | | | | |
|---|---|---|---|---|---|---|---|---|---|---|
| | 1 | | 2 | | 3 | | 4 | | 5 | |
| | Time of day: | | | | | | | | | |
| | AM | PM | AM | PM | AM | PM | AM | PM | AM | PM |
| Oxycodone dose | 9 | 9 | 17.8 | 17.8 | 23.7 | 23.7 | 23.7 | 23.7 | 23.7 | — |
| (mg/kg i.p.) | | | | | | | | | | |

Testing involved placing mice individually into a 20 (l)×20 (w)×30 (h) cm arena for 30 min. Sessions were captured via a side view high speed (120 fps), high resolution (4K) camera. Number of jumps and total duration of paw tremors were scored from the videos by an experienced experimenter blind to conditions.

Data were analysed by SPSS using oneway ANOVA and planned contrasts.

Jumping

Data for jumping are shown in Table 5 and FIG. 2A.

TABLE 5
Raw data for FIG. 2A, showing number of jumps
for each mouse in the 30 min test session

| | |
|---|---|
| Vehicle condition mice | Oxycodone condition mice |

CMPD1-2HCL dose (mg/kg i.p.)

| 0 | 0 | 2.5 | 5 | 10 |
|---|---|---|---|---|
| 0 | 99 | 92 | 136 | 51 |
| 0 | 53 | 68 | 67 | 34 |
| 0 | 107 | 86 | 51 | 25 |
| 0 | 35 | 52 | 35 | 120 |
| 11 | 163 | 58 | 61 | 17 |
| 11 | 69 | 50 | 48 | 33 |
| 1 | 125 | 31 | 48 | 33 |
| 0 | 118 | 50 | 30 | 26 |

The overall ANOVA assessing jumping was significant [F4,35=10.51, p<0.0001]. Planned contrasts revealed mice undergoing oxycodone withdrawal jumped significantly more times during the test session [VEH 0CMPD1 vs OXCD 0CMPD1, p<0.000001].

CMPD1-2HCL significantly inhibited oxycodone withdrawal-induced jumping at all doses tested [OXCD 0CMPD1 vs: OXCD 2.5CMPD1, p=0.023; OXCD 5CMPD1, p=0.018; OXCD 10CMPD1, p<0.001].

The CMPD1-2HCL treated mice still showed elevated jumping relative to mice not undergoing oxycodone withdrawal [VEH 0CMPD1 vs: OXCD 2.5CMPD1, p=<0.001; OXCD 5CMPD1, p<0.001; OXCD 10CMPD1, p=0.011].

Paw Tremors

Data for paw tremors are shown in Table 6 and FIG. 2B.

TABLE 6
Raw data for FIG. 2B, showing total duration (seconds) of
paw tremors for each mouse in the 30 minute test session

| | |
|---|---|
| Vehicle condition mice | Oxycodone condition mice |

CMPD1-2HCL dose (mg/kg i.p.)

| 0 | 0 | 2.5 | 5 | 10 |
|---|---|---|---|---|
| 0 | 23.4 | 0.2 | 3.2 | 3.3 |
| 0 | 5.2 | 0.4 | 17 | 9.6 |
| 0 | 35.1 | 12.4 | 8.2 | 9.9 |
| 0 | 9 | 2.3 | 21.4 | 0 |
| 0 | 14.3 | 78.4 | 3 | 0 |
| 0 | 31.3 | 48 | 18.5 | 0 |
| 0 | 15.4 | 27.1 | 3 | 6 |
| 0 | 47.9 | 20.4 | 2.1 | 21.5 |

The overall ANOVA assessing paw tremors was significant [F4,35=3.97, p<0.01]. Planned contrasts revealed mice undergoing oxycodone withdrawal had paw tremors for significantly longer duration during the test session [VEH 0CMPD1 vs OXCD 0CMPD1, p<0.01]. The highest dose of CMPD1-2HCL significantly inhibited oxycodone withdrawal-induced paw tremors [OXCD 0CMPD1 vs: OXCD 2.5CMPD1, p=0.898; OXCD 5CMPD1, p=0.083; OXCD 10CMPD1, p=0.033]. Mice treated with 5 and 10 mg/kg CMPD1-2HCL no longer had significantly elevated paw tremors relative to mice not undergoing oxycodone withdrawal [VEH 0CMPD1 vs: OXCD 2.5CMPD1, p=<0.01; OXCD 5CMPD1, p=0.204; OXCD 10CMPD1, p=0.4].

### Conclusions

CMPD1-2HCL does not supress locomotor activity in an open field locomotor test at the doses used in this study (Experiment 1). CMPD1-2HCL dose dependently inhibited the dysphoric and somatic symptoms (jumping and paw tremors) of oxycodone withdrawal in Experiment 2. Overall, CMPD1-2HCL shows pronounced and consistent treatment effects on withdrawal induced jumping (a physical and/or somatic symptom of opioid withdrawal and escape behaviour that reflects the intense dysphoric state induced by opioid withdrawal) and paw tremors (another somatic symptom induced by opioid withdrawal).

### Example 2

In this example, the ability of Compound 1 to inhibit naloxone-precipitated opioid withdrawal-induced c-fos protein expression in the brain was studied. C-fos is a protein marker of neural activation. Compound 1 was administered in dihydrochloride salt form (CMPD1-2HCL).

Method

N=40 male C57BL/6 mice were assigned to one of the following four conditions:

(1) VEH, VEH (n=10)

(2) VEH, CMPD1-2HCl (n=10)

(3) OXY, VEH (n=10)

(4) OXY, CMPD1-2HCl (n=10)

Mice in the oxycodone (OXY) conditions received i.p. injections of oxycodone for 9 days with increasing doses of 9, 17.8, 23.7 and 33 mg/kg (twice daily on days 1-8, with dose increasing every other day). The morning and afternoon doses were separated by 7 h. Mice in the vehicle condition (VEH) received injections of vehicle saline instead of oxycodone. One-hour-and-forty-five minutes after the morning injection on day 9, mice were administered their i.p. dose of CMPD1-2HCL (10 mg/kg at an injection volume of 10 mg/ml) or VEH. Fifteen minutes later they received an i.p. injection of 10 mg/kg naloxone (OXY groups) or saline (VEH groups) to precipitate withdrawal.

75 minutes after their naloxone or saline injection, mice were euthanised by sodium pentobarbitone overdose, and intracardiac puncture perfusion fixation was performed with 4% paraformaldehyde, and brains were then collected for immunohistochemical processing.

Data were analysed by SPSS using two-way ANOVA and planned contrasts.

### Results

8 brain regions were identified where there was a statistically significant interaction between CMPD1-2HCL treatment and opioid withdrawal and where administration of CMPD1-2HCL also significantly inhibited withdrawal induced neural activation.

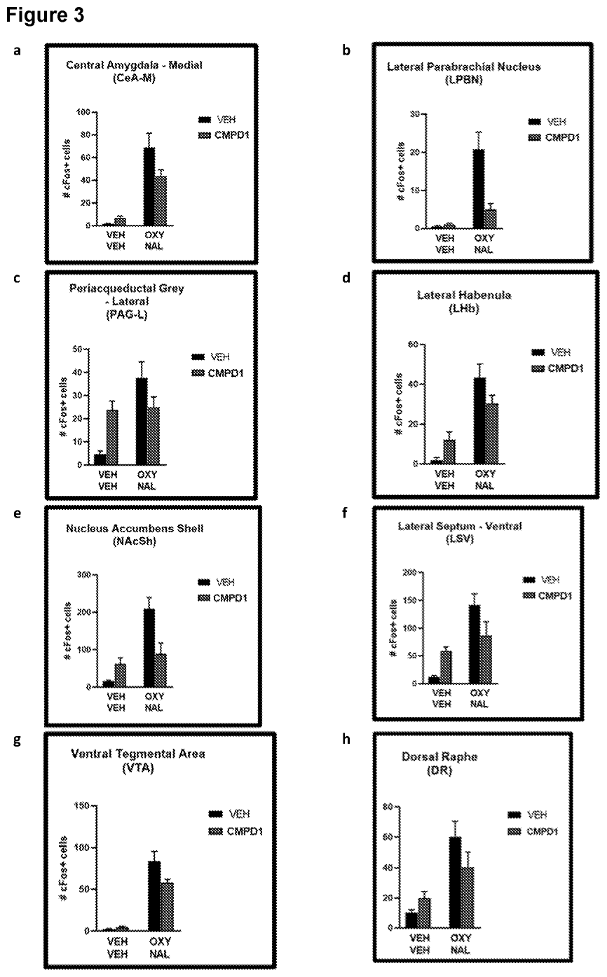

The results of the c-fos counts from these brain regions are presented in FIGS. 3a-h and include brain regions known to play a key role in the expression of opioid withdrawal symptoms.

### Example 3

In this example, the ability of CMPD1-2HCL to inhibit the gastrointestinal symptoms of opioid withdrawal was assessed using a murine model.

### Method

Adult male C57BL/6 mice (N=30) were allocated to one of four conditions (n=10 per condition):

(1) VEH, VEH (n=10)

(2) OXY, VEH (n=10)

(3) OXY, CMPD1-2HCL (n=10)

Mice in the oxycodone conditions (OXY) received i.p. injections of oxycodone for 5 days according to the schedule and doses set out in Table 7. The morning and afternoon doses were separated by 7 h. Mice in the vehicle condition received injections of vehicle saline instead of oxycodone. One-hour-and-forty-five minutes after the morning injection on day 5, mice were administered their i.p. dose of CMPD1-2HCL (10 mg/kg). Fifteen minutes later they received an i.p. injection of 10 mg/kg naloxone (oxycodone groups) or saline (vehicle group), and proceeded immediately to testing.

TABLE 7
Oxycodone dosing schedule for mice in the oxycodone conditions.

| | Day: | | | | | | | | | |
|---|---|---|---|---|---|---|---|---|---|---|
| | 1 | | 2 | | 3 | | 4 | | 5 | |
| | Time of day: | | | | | | | | | |
| | AM | PM | AM | PM | AM | PM | AM | PM | AM | PM |
| Oxycodone dose | 9 | 9 | 17.8 | 17.8 | 23.7 | 23.7 | 23.7 | 23.7 | 23.7 | — |
| (mg/kg i.p.) | | | | | | | | | | |

Testing involved placing mice individually into a 20 (l)×20 (w)×30 (h) cm arena for 30 min. The number of fecal boli were counted at the end of the session and whether or not the mouse had diarrhea.

Data were analysed using ANOVA and planned contrasts for fecal boli and Fisher's exact test for diarrhea.

Fecal Boli

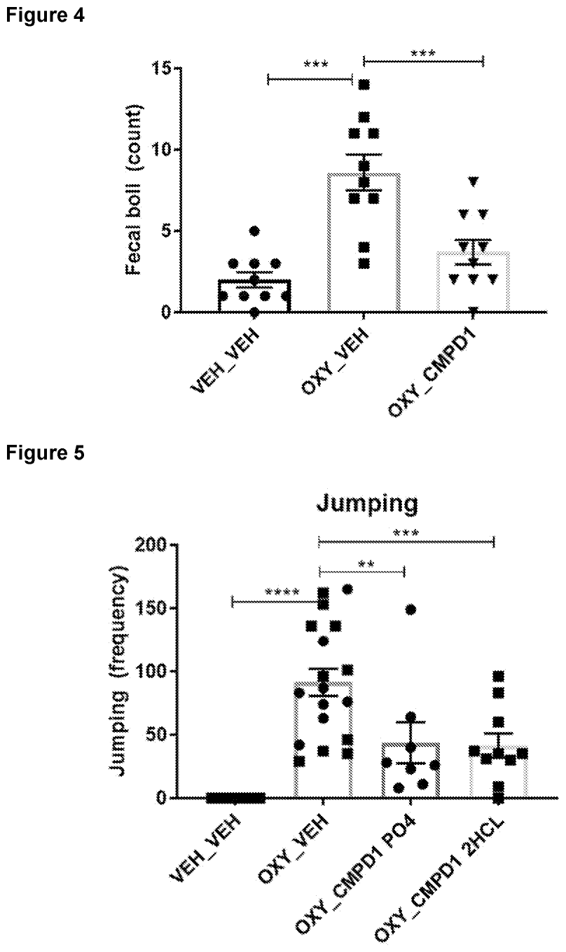

See FIG. 4. Opioid withdrawal significantly increased the amount of fecal boli produced during the 30 min session (p<0.001) and this was significantly inhibited by CMPD1-2HCL treatment (p<0.001).

Diarrhea

Opioid withdrawal significantly increased the number of mice suffering from diarrhea and this was inhibited by CMPD1-2HCL, see Table 8.

TABLE 8
Proportion of mice in each condition suffering from diarrhea.

| VEH, VEH | OXY, VEH | OXY, CMPD1-2HCL |
|---|---|---|
| 0% | 90% | 30% |

### Example 4

This Example describes experiments in a C57BL/6 mouse model of opioid withdrawal (naloxone precipitated withdrawal following oxycodone administration) and the potential of Compound 1 in two different salt forms, administered at the same freebase equivalent dose, to treat withdrawal symptoms.

### Experiment 2.1: Assessing the Effects of CMPD1-2HCL and CMPD1-PO4 on Naloxone Precipitated Oxycodone Withdrawal

Two cohorts of adult male C57BL/6 mice were run assessing the effects of 7.3 mg/kg freebase equivalent (FBE) IP Compound 1 in the dihydrochloride salt form (CMPD1-2HCL) and the phosphoric acid addition salt form (CMPD1-PO4) on naloxone precipitated withdrawal-induced jumping.

The first cohort of mice (N=30) were split into the following conditions:

(1) Vehicle, 0 mg/kg (n=10);

(2) oxycodone, 0 mg/kg Compound 1 FBE in 2HCl salt form (n=10);

(3) oxycodone, 7.3 mg/kg Compound 1 FBE in 2HCl salt form (n=10).

The second cohort of mice (N=24) were split into the following conditions:

(1) Vehicle, 0 mg/kg (n=8);

(2) oxycodone, 0 mg/kg Compound 1 FBE in phosphoric acid addition salt form (n=8);

(3) oxycodone, 7.3 mg/kg Compound 1 FBE in phosphoric acid addition salt form (n=8).

Mice in the oxycodone conditions received i.p. injections of oxycodone for 5 days according to the schedule and doses set out in Table 9. The morning and afternoon doses were separated by 7 h. Mice in the vehicle condition received injections of vehicle saline instead of oxycodone. One-hour-and-forty-five minutes after the morning injection on day 5, mice were administered their i.p. dose of Compound 1. Fifteen minutes later they received an i.p. injection of 10 mg/kg naloxone (oxycodone groups) or saline (vehicle group), and proceeded immediately to testing.

TABLE 9
Oxycodone dosing schedule for mice in the oxycodone conditions.

| | Day: | | | | | | | | | |
|---|---|---|---|---|---|---|---|---|---|---|
| | 1 | | 2 | | 3 | | 4 | | 5 | |
| | Time of day: | | | | | | | | | |
| | AM | PM | AM | PM | AM | PM | AM | PM | AM | PM |
| Oxycodone dose | 9 | 9 | 17.8 | 17.8 | 23.7 | 23.7 | 23.7 | 23.7 | 23.7 | — |
| (mg/kg i.p.) | | | | | | | | | | |

Testing involved placing mice individually into a 20 (l)×20 (w)×30 (h) cm arena for 30 min. Sessions were captured via a side view high speed (120 fps), high resolution (4K) camera. Number of jumps were scored from the videos by an experienced experimenter blind to conditions.

Data were analysed by SPSS using oneway ANOVA and planned contrasts.

Jumping

Data for jumping are shown in FIG. 5. Data from Cohort 1 mice are shown with square symbols, data from Cohort 2 mice are shown with circle symbols.

The overall ANOVA assessing jumping was significant [F3,50=21.52, p<0.0001]. Planned contrasts revealed mice undergoing oxycodone withdrawal jumped significantly more times during the test session [VEH_VEH vs OXY_VEH, p<0.0001].

7.3 mg/kg FBE Compound 1 in both the H3PO4 (CMPD1-PO4) and 2HCl (CMPD1-2HCL) addition salt form significantly inhibited oxycodone withdrawal-induced jumping at all doses tested [OXY_VEH vs: OXY_CMPD1-PO4, p<0.01; OXY_CMPD1-2HCL, p<0.001]. Moreover, the results following dosing FBE of the two salt forms did not differ significantly from each other in withdrawal-induced jumping [OXY_CMPD1-PO4 vs OXY_2 HCL, p=0.901] (see FIG. 5).

1. A method of treating opioid withdrawal or a symptom associated with the opioid withdrawal, comprising administering to a subject in need thereof an effective amount of a compound according to formula (I) 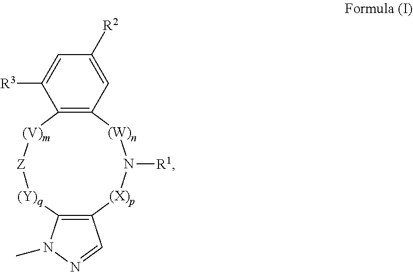
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.
or a pharmaceutically acceptable salt and/or prodrug thereof.

2. The method of claim 1, wherein the compound of Formula (I) is a compound of Formula (Ia), 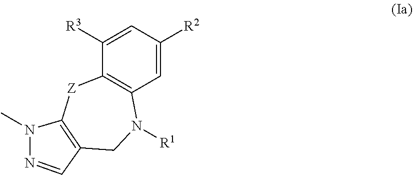
wherein:
Z is selected from: NH, O, S, S(O) or SO₂;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a pharmaceutically acceptable salt and/or prodrug thereof.

3. The method of claim 1 or 2, wherein the compound of Formula (I) is 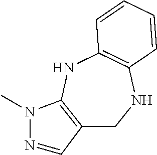
or a pharmaceutically acceptable salt and/or prodrug thereof.

4. The method of any one of claims 1-3, wherein the subject has been exposed to an opioid and/or opiate, and administration of the compound of Formula (I) or a pharmaceutically acceptable salt and/or prodrug thereof is initiated after cessation of the opioid and/or opiate exposure.

5. The method of any one of claims 1-3, wherein the subject has been exposed to an opioid and/or opiate, and administration of the compound of Formula (I) or a pharmaceutically acceptable salt and/or prodrug thereof is initiated concurrently with the last dose of the opioid and/or opiate.

6. The method of any one of claims 1-3, wherein the subject has been exposed to an opioid and/or opiate, and administration of the compound of Formula (I) or a pharmaceutically acceptable salt and/or prodrug thereof is initiated prior to cessation of opioid and/or opiate exposure.

7. The method of any one of claims 1-6, wherein the method further comprises administering an opioid and/or opiate according to a tapering dosage regimen.

8. The method of claim 7, wherein administering the compound of Formula (I) or a pharmaceutically acceptable salt and/or prodrug thereof is initiated prior to commencement of opioid/opiate dose tapering.

9. The method of claim 7, wherein administering the compound of Formula (I) or a pharmaceutically acceptable salt and/or prodrug thereof is initiated concurrently with commencement of opioid/opiate dose tapering.

10. The method of claim 7, wherein administering the compound of Formula (I) or a pharmaceutically acceptable salt and/or prodrug thereof is initiated following conclusion of opioid/opiate dose tapering.

11. The method of any one of claims 1-10, wherein administration of the compound of Formula (I) or a pharmaceutically acceptable salt and/or prodrug thereof is maintained for at least about 1 week.

12. The method of any one of claims 1-11, wherein the symptom is selected from the group consisting of tremors, shaking, hot or cold flashes, goosebumps, sweating, rapid breathing, elevated heart rate, elevated blood pressure, body aches, vomiting, diarrhea, fever, dysphoria, anxiety, restlessness, irritability, insomnia, yawning, hallucinations, hyperalgesia, hyperkatifiteia, and anorexia, or a combination thereof.

13. Use of a compound of Formula (I) 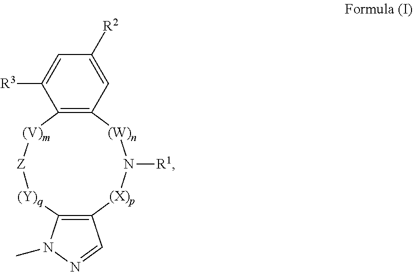
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1
or a pharmaceutically acceptable salt or prodrug thereof
in the preparation of a medicament for treating opioid withdrawal and/or a symptom associated with the opioid withdrawal.

14. A compound of Formula (I) for use in treating opioid withdrawal and/or a symptom associated with the opioid withdrawal, 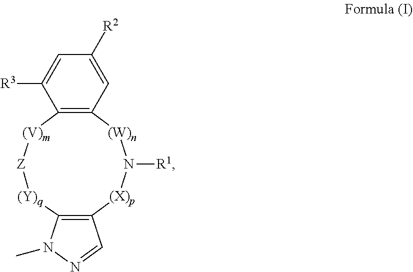
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1
or a pharmaceutically acceptable salt and/or prodrug thereof.

15. A kit comprising in separate parts:
a compound of Formula (I) as defined in claim 1 or a pharmaceutically acceptable salt and/or prodrug thereof; and
an opioid and/or opiate.

16. The kit of claim 15, wherein the opioid and/or opiate is/are provided in a plurality of dosage forms suitable for a tapering regimen.

17. A pharmaceutical composition comprising:
an effective amount of a compound of Formula (I) as defined in claim 1 or a pharmaceutically acceptable salt and/or prodrug thereof; and
an opioid and/or opiate.

18. A kit comprising in separate parts:
a compound of Formula (I) as defined in claim 1 or a pharmaceutically acceptable salt and/or prodrug thereof; and
an opioid antagonist and/or partial agonist.

19. A pharmaceutical composition comprising:
an effective amount of an opioid antagonist and/or partial agonist; and
an effective amount of a compound of Formula (I) as defined in claim 1 or a pharmaceutically acceptable salt or prodrug thereof.
